(19) United States
(12) Patent Application Publication
(10) Pub. No.: US 2022/0401413 A1
(43) Pub. Date: Dec. 22, 2022

(54) APREMILAST LOW-DOSE TOPICAL PHARMACEUTICAL COMPOSITIONS

(71) Applicant: SARUDBHAVA FORMULATIONS PRIVATE LIMITED, Hyderabad (IN)

(72) Inventors: Venkata Nookaraju SREEDHARALA, Hyderabad (IN); Srikanth KALAKOTI, Hyderabad (IN)

(73) Assignee: SARUDBHAVA FORMULATIONS PRIVATE LIMITED, Hyderabad (IN)

(21) Appl. No.: 17/774,981

(22) PCT Filed: Nov. 20, 2020
(86) PCT No.: PCT/IB2020/060949

§ 371 Date: May 6, 2022

(30) Foreign Application Priority Data
Nov. 6, 2019 (IN) 201941044971

(51) Int. Cl. A61K 31/4035; A61K 9/00; A61K 45/06; A61K 9/06; A61K 47/02

(52) U.S. Cl. CPC A61K 31/4035; A61K 9/0014; A61K 45/06; A61K 9/06; A61K 47/02

(57) ABSTRACT

The present invention provides a low dose topical composition of Apremilast with pharmaceutically acceptable excipients. The present invention also relates to a topical clear gel composition of apremilast for delivering a desired therapeutic amount of drug to the site of action after topical application. The present invention also provides a process for preparation process for preparation of low dose apremilast topical composition with enhanced diffusion and dose-proportionate linear drug release from the composition.

### CROSS REFERENCE

This application claims priority to an Indian provisional patent application no. 201941044971, filed Nov. 6, 2019, the contents of which are incorporated herein by reference in their entireties.

### FIELD OF INVENTION

The present invention relates to low dose topical clear gel compositions of Apremilast with dose proportionate diffusion of drug through the skin. The present invention is also related to process for preparation of low dose topical composition of Apremilast with linear and enhanced diffusion.

### BACKGROUND

Apremilast is chemically known as N-[2-[(1S)-1-(3-ethoxy-4-methoxyphenyl)-2-(methylsulfonyl)ethyl]-1,3-dioxo-2,3-dihydro-1H-isoindol-4-yl]acetamide which can be characterized by the following chemical formula:

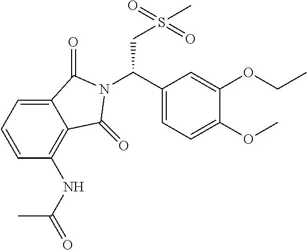 [SMILES: CCOc1cc([C@@H](CS(C)(=O)=O)N2C(=O)c3cccc(NC(C)=O)c3C2=O)ccc1OC]

Apremilast is a selective inhibitor of the enzyme phosphodiesterase 4 (PDE4) and inhibits spontaneous production of TNF-alpha from human rheumatoid synovial cells. Apremilast being a selective phosphodiesterase 4 (PDE4) inhibitor it acts by increasing the intracellular cyclic adenosine mono-phosphate (cAMP) which decreases the expression of inflammatory cytokines such as tumour necrosis factor and interleukin-23 (IL-23). It is indicated for the treatment of active psoriatic arthritis and plaque psoriasis. Commercially available forms of Apremilast are available as tablets of 10, 20 and 30 mg for oral administration. Apremilast is approved in United States of America and Europe, marketed by Celgene Corp as OTEZLA®.

U.S. Pat. No. 6,020,358 discloses the Apremilast compound and the process for its preparation. Various oral dosage forms of Apremilast are known, U.S. Pat. Nos. 7,427,638, 9,351,957, US patent application no. 20130164376, US patent application no. 20150306226 disclose different oral dosage forms. U.S. Pat. No. 9,872,854 discloses orally administered Apremilast for treatment of psoriasis with other topical agents in combination with oral Apremilast.

However oral dosage forms have sufficient evidential side effects like diarrhoea, nausea and headache. Besides these common side effects, other gastrointestinal disorders are also observed with oral Apremilast. General, oral route is a suboptimal method to treat diseases of the skin. Particularly the systemic administration of the drug (via the oral route or otherwise) carries risk of side effect in tissues unconnected with such conditions. To overcome oral drugs first pass metabolism by the liver and side effects thereof, topical administration would frequently be the route of choice, if it could be achieved. Various publications viz., WO 2018/138737 discloses topical compositions of Apremilast with specific alcoholic, aqueous carriers and excipients. WO 2017/216738 discloses topical composition of Apremilast free from alpha-hydroxy acids, with specific ratio of penetration enhancer/solubilizer. CN105168136 discloses an Apremilast preparation with cholesterol and phospholipids as essential ingredients. WO 2017/168433 discloses an Apremilast topical composition with specific drug to carrier ratio. CN108283620A discloses a topical composition of Phosphodiesterase inhibitor with DMSO and carrier.

Therefore there are few literatures available for topical Apremilast composition. PCT publication number WO2018138737A1 discloses formulations for the topical administration of Apremilast 1-20% for management of psoriasis and other dermatological conditions. PCT publication number WO2017216738A1 discloses topical pharmaceutical compositions comprising Apremilast in an amount of about 0.1 to 5% w/w of the total composition and solubilizers and process for their preparation. The composition disclosed has solubilizers and penetration enhancers. PCT publication number WO2017168433A1 discloses topical pharmaceutical compositions of Apremilast in concentration range of 0.5-25% with carrier DMSO for the treatment of psoriasis and/or psoriatic arthritis. The discloser also uses variety of excipients in the compositions. PCT publication number WO2009120167A1 discloses various solid forms of Apremilast, method of preparation and their use in various skin conditions including psoriasis in daily dose of 1 to 1000 mg per day orally. United States patent application publication number US20190060221A1 discloses topical pharmaceutical composition of Apremilast or its pharmaceutical acceptable salts in range of 0.01 to 25% w/w with isopropyl myristate and process for the preparation thereof. All the composition disclosed in literature even though claim a wider range of drug viz., 0.01-25%, there is no support for actual workable or enabling composition in the full claimed ranges, more specifically, workability of the composition below 1% of drug concentration is unpredictable and feasibility of such composition is also difficult in view of the solubility of Apremilast.

Further though there are literatures available disclosing various topical compositions of Apremilast, there is a critical issue in such composition which is needed to be addressed. The formulations depicted in all the literatures, particularly in WO2018138737 A1 discloses a dose-dependent increase in efficacy between 2% and 4%; however a saturation of efficacy was observed at 10% concentration of Apremilast. This observation clearly suggests that for topical applications the therapeutic saturation is very critical and of concern. Further, it is expected that with continuous topical application of a higher concentration of drug on the same (affected) site, skin tissue binding and uptake may become saturated, with greater portions of the later topical doses being systemically absorbed (William R. Ravis Et al., Antimicrobial Agents and Chemotherapy p. 4809-4815, October 2013 Volume 57 Number 10).

The major rationale behind development of a topical composition of Apremilast is to reduce systemic effects of Apremilast and to treat psoriasis and other related inflammatory skin conditions. Topical Apremilast composition particularly as in prior art literature WO2018138737, WO2017168433A1, WO2009120167A1, US20190060221A1, all sooner or later may cause saturation of the dermal tissue thereby causing therapeutic saturation and gradual systemic absorption (William R. Ravis Et al.), which will ultimately in long run fail the purpose of topical treatment of psoriasis and may cause potential systemic adverse events and prevent their optimal long-term and extensive utilization.

Thus despite the presence of various topical compositions there is need in the art for a stable and highly effective, convenient composition which can provide controlled/sustained release of the drug and is consistent and non-greasy on skin over conventional topical compositions and most importantly should not cause any systemic absorption, as the amount of drug used should be very minimal or less in concentration. Also the key aspect for topical composition is penetration or diffusion into the skin layers for better efficacy, but penetration at systemic level may cause various other side effects as the course of treatment is longer and require a chronic therapy. Psoriasis being a lifelong, chronic and immune-mediated systemic disease, the different presentations of psoriasis requires different approaches to treatment and appropriate prescriptions according to disease severity. The use of topical therapy remains a key component of the management of almost all psoriasis patients.

Moreover from formulator's point of view due to the insolubility of Apremilast itself in water and minimal solubility in polar solvents, it is challenging to formulate a proper topical composition of Apremilast with a lower amount of drug and having better and proportional diffusion/penetrating capabilities, providing an effective concentration of Apremilast at the site of action for a longer period of time to give enhanced therapeutic results without any systemic side effects or drug saturation at the site of application. Further a general perception exists that a reduced dose would not deliver the desired effect, and an excessive dose may lead to undesirable side effects. The delivery of the correct dose of the drug depends highly on the spreadability of the formulation and the response of the target site to the amount of drug reaching the site.

The inventors of the present invention have surprisingly found that topical composition of Apremilast as clear gels in comparison with other prior art topical formulation that can lead to significant dose reduction, which not only provides improved efficacy because of site specific availability of desired concentration of drug as well as minimize the adverse reactions associated with higher amount of drug in topical dosage formulation which usually cause resistance or saturation of therapeutic effect. Besides these, it also provides improved patient compliance and enhanced psychological impact towards therapy. The topical compositions of the invention can be used for psoriasis and related skin conditions diseases or disorders such as dermatosis, eczema, rosacea, acne vulgaris, allergies, contact and atopic dermatitis, pruritus, seborrhoea, skin cancers, inflammation and others.

### SUMMARY OF THE INVENTION

The present invention relates to a low dose topical composition of Apremilast for linear and proportional drug release.

A further object of the invention is to develop a topical composition of Apremilast for delivering a desired therapeutic amount of drug to the site of action after topical application.

Yet another object of the invention is to formulate a process for preparation of low dose Apremilast topical composition with enhanced diffusion and reduced systemic absorption or drug saturation at the site of application.

Another object of present invention is to provide a longer acting controlled release topical composition of Apremilast with enhanced skin penetration and feel-good consistency.

In a further object the present invention provides a topical pharmaceutical composition suitable for topical application, comprising Apremilast or its pharmaceutically acceptable salt in lower amount capable of delivering therapeutically effective amounts in combination with pharmaceutically acceptable excipients.

In a preferred embodiment the present invention provides a low dose clear gel of Apremilast for treatment of Psoriasis and related inflammatory skin conditions.

A further object of the invention is to provide topical compositions like cream, gel, ointment, lotion or paste which is characteristically long acting, and provide enhanced drug delivery and skin permeation with minimal amount of Apremilast in the dosage form. In another embodiment, the application provides processes for preparing such pharmaceutically stable topical compositions comprising lower amount of Apremilast.

In yet another object the present invention relates to an effective topical composition of Apremilast in lower amounts for treatment or amelioration of diseases such as psoriasis or psoriatic arthritis, dermatosis, eczema, rosacea, acne, contact and atopic dermatitis, pruritus, inflammation and other associated skin conditions.

### BRIEF DESCRIPTION OF DRAWING

FIG. 1, shows cumulative drug release from Apremilast topical gel, wherein the drug release is linear with increased strength until drug is in solution form (clear gel) whereas the drug release is more or less constant in dispersive gels irrespective of strengths.

### DETAILED DESCRIPTION

The inventors of the present invention are consistently working towards various approaches for developing compositions for better and effective remedy. In line with the ongoing research, the inventors have surprisingly found out that there is significant enhanced penetration of the drug or diffusion of the drug (Apremilast), when used in lower amounts i.e., the drug release is linear with increased strengths until drug is in solution (as in clear gels) form. Thus the inventors surprisingly found that at lower amounts of drug the topical composition has proportional increase in diffusion rate. However with gradual increase in the amount of drug the diffusion rate is almost constant and leads to a flat effect, this may be attributed to precipitation of the drug or Apremilast in the dispersive gels, or may be due to the saturation of the diffusion layer mimicking the skin.

The present disclosure is herein described in detail with reference to embodiments, which form a part here. Other embodiments may be used and/or other changes may be made without departing from the spirit or scope of the present disclosure. The illustrative embodiments described in the description are not meant to be limiting of the subject matter presented here.

The present invention pertains to topical composition of Apremilast containing lower amount of drug, various formulation were further evaluated with other components or excipients in order to obtain better acting and effective compositions.

The topical composition in the present invention is able to give proportionate increase in the amount of drug getting diffused across the diffusion layer, throughout the range of 0.01% to 1.5%.

In another embodiment the present invention relates to a lower amount of Apremilast in combination with is a plurality of excipients in specific quantity and combinations, for both solubilizing the active agent and forming a long acting, better penetrating consistent topical clear gel composition.

In another embodiment, the present invention provides a low dose topical gel or can be formulated as low dose topical cream, ointment, emulsion or lotion of Apremilast with at least one pharmaceutically acceptable excipient and having proportionate and enhanced skin diffusion/penetration, with better patient compliance, consistency and therapy.

In another embodiment, the present invention also provides a low dose topical composition of Apremilast with at least one pharmaceutically acceptable excipient and having linear and dose proportionate enhanced diffusion/penetration, with better patient compliance, consistency and therapy.

In yet another preferred embodiment the present invention provides a method of treating psoriasis or related inflammatory skin disorders such as dermatosis, eczema, rosacea, acne vulgaris, allergies, contact and atopic dermatitis, pruritus, seborrhoea, skin cancers, inflammation and others using a low dose composition of Apremilast in the range of 0.01 to 1.5% w/w of Apremilast.

In yet another embodiment the pharmaceutical compositions in the form of topical gels, creams, ointments, and lotions as prepared in the present invention provide desired pharmacological actions and fewer side effects, along with better patient compliance.

In yet another preferred embodiment the low dose topical composition of Apremilast in the present invention is able to provide an effective amount of drug at the site of action for treatment of mild to major psoriasis and is efficient in reducing the psoriatic scores in effective manner.

In one preferred embodiment of the invention, the Apremilast provided in the clear topical gel which is better than the dispersion gels, thereby comparatively giving a linear and proportionate diffusion of drug when compared with later.

In another embodiment of the invention, Apremilast is provided as a topical pharmaceutical composition with pharmaceutically acceptable carriers, solvents, solubilizers, humectants, penetration enhancers, surfactants, preservatives, gelling agents, co-solvents and other topically acceptable excipients.

In yet another embodiment of the invention, topical gel may comprise excipients that are hydrophilic and/or hydrophobic in nature.

In another preferred embodiment of the invention, the topical composition comprises of Apremilast or its pharmaceutically acceptable salt and a suitable solvent, solubilizer, a suitable carrier and penetration enhancer with gelling agent in suitable ratio, to give maximum average flux or diffusion of the drug through the skin layers.

Unless otherwise recited or required by the context, percent and “%” refer to percent by weight.

Topical pharmaceutical compositions of the invention include Apremilast in the concentration of 0.01% to 1.5% by weight, preferably 0.01 to 1% w/w, more preferably 0.05-0.5% w/w.

Topical pharmaceutical compositions of the invention include Apremilast and gelling agent in an amount of 0.01-1.5% w/w and 0.05-5% w/w respectively or in the ratio of 1:10 to 1:100 respectively.

Topical pharmaceutical compositions of the invention have a pH in the physiological range between 4 to 8 or 4.5 to 6.5.

In yet another preferred embodiment the ratio of drug to the solvent in the composition is ranging from 0.01:100 to 100:0.01 or 0.1:50 to 50:0.1 or 0.01:5 to 0.5:50.

In yet another preferred embodiment the ratio of drug to the co-solvent in the composition is ranging from 0.01:100 to 100:0.01 or 0.1:50 to 50:0.1 or 0.01:5 to 0.5:50.

In yet another embodiment the penetration enhancer or solubilizer in the composition can be a single solubilizer/surfactant or a combination of solubilizer/surfactants, combined in such a concentration to impart maximum drug diffusion through the skin.

In yet another embodiment the solvents used in the invention can have multiple application like a solvent can be used as co-solvent, penetration enhancer or solubilizer in the composition and can be a single solvent or a combination of solvent to play different roles as Solubilizers/penetration enhancer/surfactants/humectant, combined in such a concentration to impart maximum and linear drug diffusion through the skin.

In another preferred embodiment, the topical clear gel composition of the invention comprises Apremilast 0.01-1.5% w/w, gelling agent 0.05-5% w/w, solubilizer 1-30% w/w, penetration enhancer 5-40%, humectant 5-40%, solvent/carrier 2-20% w/w, preservative 0.05-5% w/w, purified water 5-80% w/w and suitable pH adjusting agent or buffering agent or alkalizer.

In another preferred embodiment, the topical clear gel composition of the invention comprises Apremilast 0.01-0.5% w/w, gelling agent 0.05-5% w/w, solubilizer 1-30% w/w, penetration enhancer 5-40%, humectant 5-40%, solvent/carrier 2-20% w/w, preservative 0.05-5% w/w, purified water 5-80% w/w and suitable pH adjusting agent or buffering agent or alkalizer.

In another preferred embodiment, the topical clear gel composition of the invention comprises Apremilast 0.01-1.5% w/w, gelling agent 0.05-5% w/w, a first solvent 1-30% w/w, a second solvent 5-40% w/w, a humectant 5-40% w/w, a third solvent 2-20% w/w, preservative 0.05-5% w/w, purified water 5-80% w/w and suitable pH adjusting agent or buffering agent or alkalizer.

In another preferred embodiment, the topical clear gel composition of the invention comprises Apremilast 0.01-0.5% w/w, gelling agent 0.05-5% w/w, a first solvent 1-30% w/w, a second solvent 5-40% w/w, a humectant 5-40% w/w, a third solvent 2-20% w/w, preservative 0.05-5% w/w, purified water 5-80% w/w and suitable pH adjusting agent or buffering agent or alkalizer.

In yet another embodiment the first, second and third solvent used in the present invention compositions can be used interchangeably in different ratios to provide various formulations with similar or varying release profiles.

In yet another embodiment of the invention, topical clear gel composition also comprises excipients to provide better feel to the skin, no greasiness or feel of any gritty particles and lower irritation to the skin.

In one of the embodiment of the invention, topical pharmaceutical composition may optionally comprise of suitable preservative or fragrance.

In one preferred embodiment, the invention is directed to a topical composition for treating psoriasis comprising a therapeutically effective amount of Apremilast, a suitable solvent or mixture thereof, a solubilizer/permeation enhancer and a pharmaceutically acceptable carrier.

In yet another preferred embodiment the process for preparation of topical clear gel composition of the invention comprises of:
a) Dispensing actual quantities of Apremilast, carrier/solvent, gelling agent, 2^{nd} solvent, humectant, methyl paraben, propyl paraben and 3^{rd} solvent.
b) Weighing the required quantity of Apremilast and dissolving in required quantity of suitable solvent/carrier (1^{st} solvent) under stirring. The stirring was continued until the complete solubilization of Apremilast.
c) Weighing required quantity of water and a suitable humectant and mixing together under continuous stirring.
d) Adding weighed quantity of 2^{nd} solvent to the step c) mixture under stirring with continuous stirring for 10 min.
e) Then suitable gelling agent was added to the step d) mixture under stirring and the stirring was continued for 2 hrs till complete solubilization of the gelling agent.
f) There after step b) drug solution was added to the step e) gel base under stirring.
g) Separately weighed quantity of methyl and propyl parabens were then dissolved in required quantity of 3^{rd} solvent.
h) Then the step g) preservative solution was added to the step f) Apremilast gel under stirring. Followed by continued stirring for 30 min.
i) Finally the pH of the gel was adjusted in the range of pH 6.0-7.0 by adding 10% w/v NaOH solution.

In the process the solvents used in step a), b), d) and g) can be same or different and can play a similar or distinct role in the topical formulation.

A “therapeutically effective amount” is an amount necessary to palliate or treat at least one symptom of psoriasis. For example, a therapeutically effective amount is sufficient to treat (i.e. alleviate or reduce) at least one of: itching/scratching, redness, inflammation, cracking, scaling, bleeding, etc. Preferably, the therapeutically effective amount of Apremilast comprises between 0.01 to 1.5% by weight of the composition, more preferably 0.05 to 0.5% w/w of the composition.

In an embodiment of the invention, topical composition of present invention has excipients that help effective diffusion/penetration of Apremilast in to the skin and provide ease of application, spreadability and cleaning.

Non-Limiting Lists of the Excipients that can be Used in the Composition are:

Typically, the topical compositions of the invention comprise various gelling agents, solvents and co-solvents, skin penetration enhancers, pharmaceutical surfactants and solubility enhancers, oil phase components, aqueous phase components, emulsifiers, moisturizers, antioxidants, vitamins, lubricants, preservatives, stabilizers, buffers, alkalizers and other ingredients. Also the components/excipients/solvents as embodied in the present composition may have more than one application.

Various gelling agents include gelling agents from synthetic and other sources, carbopol and its derivatives and any other pharmaceutically acceptable gelling agents, further gelling agents include, but are not limited to gums, acrylic acid and acrylate polymers and. copolymers, and cellulose derivatives (e.g., hydroxymethyl cellulose and hydroxypropyl cellulose), one high molecular weight, cross linked polyacrylic acid polymer, Carbopol 10 NF. Pharmaceutically acceptable water-gelling agents preferably are carbomers, glyceryl polyacrylates, and combinations thereof. Various viscosity grades sold under the trademark designation CARBOPOL® by B.F. Goodrich Company, Cleveland, Ohio. Particularly preferred is CARBOPOL® 980. Glyceryl polyacrylates are esters of glycerine and polyacrylic acid available in various viscosity grades sold as an aqueous jelly under the trademark designation, HISPAGEL, by Hispano Quimica S. A., Barcelona, Spain.

Skin penetration enhancers reversibly decrease the barrier resistance of the skin, which increases the amount of Apremilast absorbed. Preferably, skin penetration enhancers include, but are not limited to, PGs (Propylene Glycols), sulfoxides (e.g. DMSO), azones (e.g. laurocapram), pyrrolidones (e.g., 2-pyrrolidone), alcohols and alkanols (e.g., ethanol, decanol, etc.), oleic acid (and derivatives thereof), glycols (e.g., propylene glycol), dimethylformamide (DMF), dimethylacetamide (DMAC), fatty alcohols (e.g., lauryl alcohol), fatty acid esters, fatty acids, fatty alcohol ethers (e.g., EO-2-oleyl ether), terpenes, and lecithin.

Pharmaceutical surfactants or solubility enhancers include, but are not limited to, lauryl alcohol, polyoxyethylene ether, polyoxyethylene glycerol monostearate, stearic acid ester oxygen poly hydrocarbon, vitamin E succinate polyethylene glycol ester, sorbitan esters, polyoxyethylene castor oil, polyoxyethylene hydrogenated castor oil, poloxamer, organic esters (e.g. ethylene acetate), and poly hill dinitrate 80 (i.e. Tween 80 or its mixture). In a preferred embodiment, the pharmaceutical surfactants or solubility enhancers include DMSO, polyvinylpyrrolidones, stearic acid hydrocarbon oxygen Poly (40) ester, lauryl alcohol polyoxyethylene (23) ether, vitamin E succinate polyethylene glycol ester, ethylene acetate, and polyoxyethylene (40) hydrogenated castor oil (and its mixtures, i.e. polyoxy (40) stearate). Still, in a more preferred embodiment the pharmaceutical surfactants or solubility enhancers include sodium lauryl sulphate and sorbitan esters.

Suitable oily phase may include, but are not limited to, glyceryl monoacetate, glycerol diacetate, glyceryl triacetate, stearic acid, various commercially available pharmaceutical grade oils, oleic acid, medium-chain triglycerides, single-decane triglyceride, fat (e.g., lanolin), mineral oils, paraffin, waxes, petrolatum, hydrocarbons, vaseline, and mixtures thereof.

Preferred non-limiting examples of hydrophobic excipients useful in the present composition include small and medium chain triglycerides, petrolatum, red petrolatum, white petrolatum, liquid petrolatum, semi-solid petrolatum, light mineral oil, heavy mineral oil, white mineral oil, mineral oil alcohols, C₇-C₄₀ branched chain hydrocarbons, C₁₀-C₃₀ alcohol esters of C₁₀-C₃₀ carboxylic acids, C₁₀-C₃₀ alcohol esters of C₁₀-C₃₀ dicarboxylic acids, monoglycerides of C₁₀-C₃₀ carboxylic acids, diglycerides of C₁₀-C₃₀ carboxylic acids, triglycerides of C₁₀-C₃₀ carboxylic acids, ethylene glycol monoesters of C₁₀-C₃₀ carboxylic acids, ethylene glycol diesters of C₁₀-C₃₀ carboxylic acids, propylene glycol monoesters of C₁₀-C₃₀ carboxylic acids, propylene glycol diesters of C₁₀-C₃₀ carboxylic acids, C₁₀-C₃₀ carboxylic acid monesters and polyesters of sugars, pharmaceutical grade oils and their hydrogenated oil forms, Shea butter, polypropylene glycols, polypropylene glycol C₄-C₂₀ alkyl ethers, di C₈-C₃₀ alkyl ethers, synthetic hydrocarbons, derivatives thereof, and mixtures thereof. Also additionally preferred, non-limiting examples of other hydrophobic agents include ethylene glycol distearate, cetyl palmitate, myristyl myristate, stearyl stearate, cetyl stearate, behenyl behenate, caprylic/capric triglyceride, PEG-6 caprylic/capric triglyceride, PEG-8 caprylic/capric triglyceride, derivatives thereof, and mixtures thereof.

Further the composition may comprise pharmaceutically acceptable carriers like water, glycerin, petrolatum, stearic acid, glycol stearate, dimethicone, isopropyl isostearate, pharmaceutical grade starch, cetyl alcohol, glyceryl stearate, magnesium aluminum silicate, carbomer, ethylene brassylate, triethanolamine, disodium EDTA, phenoxyethanol, methyl paraben, propyl paraben, ethanol, bio-polymers (e.g., sodium hyaloronate), liposomes, nano- and micro-particulate carriers, and/or titanium dioxide. More preferably, the pharmaceutically acceptable carrier comprises dimethyl sulfoxide (DMSO), glycerol, propylene glycol, petrolatum water, and one or more pharmaceutically acceptable penetration enhancer (absorption promoter and/or accelerants). Aqueous phase components include, but are not limited to, de-ionized water, glycerol gelatin, cellulose derivatives (e.g., microcrystalline cellulose (Avicel PH 101)), and polyethylene glycol (PEG 300 to PEG 6000), and mixtures thereof.

Emulsifiers include, but are not limited to Emulcire 61 WL 1349, Gelot 64, oleyl alcohol, polyoxyethylene oleyl ether, PEG-40 stearate, ceteareth-12, ceteareth-20, ceteareth-30, glyceryl stearate, PEG-100 stearate, methyl myristate, isopropyl myristate, Arlacel 165, glyceryl stearate, PEG-100 stearate, steareth-2 and steareth-20, dimethicone copolyol, Polysorbate 20 (Tween 20), cetyl esters wax, Polysorbate 40 (Tween 40), Polysorbate 60 (Tween 60), Polysorbate 80 (Tween 80), lauramide DEA, cocamide DEA, and cocamide MEA, Phospholipid PTC, alginate, carrageenan, Glucate DO, methylcellulose, polyvinyl alcohol, Carbopol and Carbomer. Preferably, emulsifiers are selected from the group consisting of cetostearyl alcohol, stearic acid, magnesium stearate, sodium lauryl sulfate, triethanolamine, and magnesium aluminum silicate.

Any other emulsifiers known to those of skill in the art as useful in the formation of topical compositions are further contemplated herein.

Moisturizers include, but are not limited to, glycerol, pentylene glycol, butylene glycol, polyethylene glycol, sodium pyrrolidone carboxylate, alpha-hydroxy acids, beta-hydroxy acids, polyhydric alcohols, ethoxylated and propoxylated polyols, polyols, polysaccharides, panthenol, hexylene glycol, propylene glycol, dipropylene glycol, sorbitol, derivatives thereof, and mixtures thereof.

Antioxidants include, but are not limited to, water soluble antioxidants, lipid-soluble antioxidants, vitamin C, vitamin C palmitate, propyl gallate, vitamin E (tocopherol), tert-butyl ether-hydroxybenzoate fennel, 2,6 di-tert-butyl-p-cresol, BHA, BHT, or mixtures of one or more antioxidants.

Lubricants include, but are not limited to, urea, magnesium stearate, sodium lauryl sulfate, polyethylene glycol, and silica gel powder.

Preservatives include, but are not limited to, chloro-m-cresol, citric acid, disodium edetate, ethoxylated alcohol, glycerin, 1,2,6-hexanetriol, methylparaben, parabens, potassium, sorbate, propyl gallate, propylene glycol, propyl paraben, sodium bisulfate, sodium citrate, butyl paraben, sodium metabisulfite, chlorocresol, sorbic acid, tannic acid, zinc stearate, butylated hydroxytoluene, butylated hydroxyanisole, benzoic acid, salicylic acid, propyl paraben, dichlorobenzyl alcohol, formaldehyde, alpha-tocopherol, sodium ascorbate, ascorbic acid, ascorbyl palmitate phenol, m-cresol, bisphenol, cetrimide, benzalkonium chloride, sorbic acid, phenoxyethanol, and benzoyl peroxide. Preferably, preservatives are selected from the group consisting of hydroxylethyl benzene, hydroxylmethyl benzene, phenoxyethanol, chlorocresol, propyl paraben, and methyl paraben.

Any other solvent/co-solvent known to those of skill in the art as useful in the formation of topical compositions are further contemplated herein. Further the solvent and co-solvent can have multiple applications like Solubilizers, penetration enhancers, humectants, moisturisers and other topically feasible roles known to a person skilled in the art.

The presently preferred compositions may further contain a pH modifier. Preferably, the presently preferred compositions can comprise about 0.01% to about 10% by weight of a pH modifier. Preferred non-limiting examples of neutralizing pH modifiers that can optionally be included in these compositions include inorganic hydroxides, inorganic oxides, inorganic salts of weak acids, derivatives thereof, and mixtures thereof.

Preferred non-limiting examples of inorganic hydroxides useful in this regard include ammonium hydroxide, alkali metal hydroxide, alkaline earth metal hydroxides, derivatives thereof, and mixtures thereof.

Preferred inorganic hydroxides useful in this regard include ammonium hydroxide, monovalent alkali metal hydroxides such as sodium hydroxide and potassium hydroxide, divalent alkali earth metal hydroxides such as calcium hydroxide and magnesium hydroxide, derivatives thereof, and mixtures thereof.

Preferred non-limiting examples of inorganic oxides useful in this regard include magnesium oxide, calcium oxide, derivatives thereof, and mixtures thereof.

Preferred, non-limiting examples of inorganic salts of weak acids useful in this regard include ammonium phosphate (dibasic), alkali metal salts of weak acids such as sodium acetate, sodium borate, sodium metaborate, sodium carbonate, sodium bicarbonate, sodium phosphate (tribasic), sodium phosphate (dibasic), potassium carbonate, potassium bicarbonate, potassium citrate, potassium acetate, potassium phosphate (dibasic), potassium phosphate (tribasic), alkaline earth metal salts of weak acids such as magnesium phosphate and calcium phosphate, derivatives thereof, and mixtures thereof.

In an embodiment of the invention, topical composition of present invention has pH adjusting agents that help in adjusting the pH of the composition between pH of about 4.5 to about 7.5 to provide a stable and non-irritating composition. Such agents include many pharmaceutically acceptable acids, bases and buffers. Suitable acids may include one or more of hydrochloric acid, phosphoric acid and lactic acid. Suitable bases may include one or more of diethanolamine, triethanolamine, triethylamine and sodium hydroxide. Suitable buffers may include phosphates, such as monobasic sodium phosphate, dibasic sodium phosphate, lactates and citrates.

### Definitions

As used here, the following terms have the following definitions:

“Active Pharmaceutical Ingredients (APIs)” refer to chemical compounds that induce a desired effect, and include agents that are therapeutically or prophylactically effective.

“Permeation enhancement” refers to an increase in the permeability of the skin or mucosal tissue to the selected active pharmaceutical ingredients.

“Topical administration” refers to delivery of a topical drug or active pharmaceutical ingredient to the skin or mucosa, thus providing a local effect.

“Treating” or “Treatment” refers to reduction in severity and/or frequency of symptoms, elimination of symptoms and/or underlying cause, prevention of the occurrence of symptoms and/or their underlying cause, and improvement or remediation of damage.

The term “pharmaceutically acceptable” means approved by a regulatory agency of the Federal or state government or listed in the U.S. Pharmacopeia or other generally recognized pharmacopeia for use in animals, and more particularly in humans.

The term “penetration enhancer” refers to vehicle, carrier, diluent, adjuvant, excipient, or solubilizer with which an active ingredient is administered. Such pharmaceutical agents can be lipophilic or hydrophobic components.

The term “carrier” refers to a diluent, adjuvant, excipient, penetration enhancer, or vehicle with which an active ingredient is administered.

### EXAMPLES

The following examples which include preferred embodiments will serve to illustrate the practice of this invention, it being understood that the particulars shown are by way of example and for purposes of illustrative discussion of preferred embodiments of the present invention only and are presented in the cause of proving what is believed to be the most useful and readily understood description of formulation procedures as well as of the principles and conceptual aspects of the invention. It will be evident to those skilled in the art that the invention is not limited to the details of the following illustrative examples and that the present invention may be embodied in other specific forms without departing from the essential attributes thereof, and it is therefore desired that the present embodiments and examples be considered in all respects as illustrative and not restrictive, reference being made to the appended claims, rather than to the foregoing description, and all changes which come within the meaning and range of equivalency of the claims are therefore intended to be embraced therein.

### Example 1: Apremilast Topical Clear Gel

| Component | % w/w |
|---|---|
| Apremilast | 0.01-1.5 |
| DMSO | 1-30 |
| Carbopol | 0.05-5 |
| Glycerin | 5-40 |
| Propylene glycol | 5-40 |
| Ethanol | 2-20 |
| Methyl paraben | 0.05-5 |
| propyl paraben | 0.05-5 |
| Purified Water | 5-80 |
| Sodium hydroxide | q.s. |
| Total | 100 |

Brief Manufacturing Process:
a) Actual quantities of Apremilast, DMSO, carbopol, glycerine, propylene glycol, methyl paraben, propyl paraben and ethanol were dispensed.
b) The Apremilast was weighed and dissolved in required quantity of DMSO under stirring. The stirring was continued until the complete solubilization of Apremilast.
c) Weighed quantity of water and propylene glycol were mixed together under continuous stirring.
d) Followed by adding weighed quantity of glycerine to the step c) mixture under stirring with continuous stirring for 10 min.
e) Then carbopol was added to the step d) mixture under stirring and the stirring was continued for 2 hrs till complete solubilization of carbopol.
f) There after step b) drug solution was added to the step e) gel base under stirring.
g) Separately weighed quantity of methyl and propyl parabens were then dissolved in required quantity of ethanol.
h) Then the step g) preservative solution was added to the step f) Apremilast gel under stirring. Followed by continued stirring for 30 min.
i) Finally the pH of the gel was adjusted to 6.0±0.5 by adding 10% w/v NaOH solution.

### Example 2: Apremilast Topical Clear Gels

| | 0.05% w/w | 0.10% w/w | 0.25% w/w |
|---|---|---|---|
| Components | gel (w/w) | gel (% w/w) | gel (% w/w) |
| Apremilast | 0.05 | 0.10 | 0.25 |
| DMSO | q.s | q.s | q.s |
| Carbopol 10NF | 0.5 | 0.5 | 0.5 |
| Glycerin | q.s | q.s | q.s |
| Propylene glycol | 29.3 | 29.3 | 29.3 |
| Ethanol USP | q.s | q.s | q.s |
| Methyl paraben | 0.2 | 0.2 | 0.2 |
| Propyl paraben | 0.2 | 0.2 | 0.2 |
| Purified Water | 29.4 | 29.4 | 29.2 |
| Total | 100 | 100 | 100 |

### Example 3: Apremilast Dispersive Gels

| | 0.75% w/w | 1% w/w | 2% w/w gel | 4% w/w gel |
|---|---|---|---|---|
| Components | gel (% w/w) | gel (% w/w) | (% w/w) | (% w/w) |
| Apremilast | 0.75 | 1 | 2 | 4 |
| DMSO | q.s | q.s | q.s | q.s |
| Carbopol 10NF | 0.5 | 0.5 | 0.5 | 0.5 |
| Glycerin | q.s | q.s | q.s | q.s |
| Propylene | 29.3 | 29.3 | 29.3 | 29.3 |
| glycol | | | | |
| Ethanol USP | q.s | q.s | q.s | q.s |
| Methyl paraben | 0.2 | 0.2 | 0.2 | 0.2 |
| Propyl paraben | 0.2 | 0.2 | 0.2 | 0.2 |
| Purified Water | 28.7 | 28.5 | 27.5 | 25.5 |
| Total | 100 | 100 | 100 | 100 |

Observation:

The gels prepared in Examples 2 and 3 as per procedure given in Example 1 above, were further evaluated for various physical parameter and in vitro drug release of gels over a period of 48 hours as given in Table 1 (with respect to Example 2 for clear gel) and Table 2 (with respect to Example 3 for dispersive gel). The observation study depicted following results as tabulated below:

TABLE 1
For composition of Apremilast in lower
amount - clear gel - Example 2

| | | | |
|---|---|---|---|
| Strength | 0.05% | 0.10% | 0.25% |
| 1 g gel contains | 500 μg API | 1000 μg API | 2500 μg API |
| Physical | Clear gel | Clear gel | Clear gel |
| observation | | | |

Physical properties

| | Clear transparent | Clear transparent | Clear transparent |
|---|---|---|---|
| Description | viscous gel | viscous gel | viscous gel |
| pH (10% | 6.03 | 6.09 | 6.09 |
| solution) | | | |
| Viscosity (cps) | 2138 | 2184 | 1997 |
| Time (Hrs) | Cumulative drug release (μg/20 ml) | | |
| 0 | 0.0 | 0.0 | 0.0 |
| 2 | 0.0 | 0.3 | 2.2 |
| 4 | 0.2 | 1.2 | 7.3 |
| 6 | 1.7 | 4.1 | 15.2 |
| 8 | 3.5 | 7.1 | 22.7 |
| 12 | 6.8 | 13.2 | 42.8 |
| 24 | 15.5 | 27.8 | 74.7 |
| 30 | 19.2 | 34.7 | 94.6 |
| 48 | 24.0 | 43.6 | 113.9 |

TABLE 2
For composition of Apremilast - Dispersive Gel - Example 3

| Strength | 0.75% | 1% | 2% | 4% |
|---|---|---|---|---|
| 1 g gel contains | 7500 μg API | 10000 μg API | 20000 μg API | 40000 μg API |
| Physical observation | API dispersed | API dispersed | API dispersed | API dispersed |
| | Physical properties | | | |
| Description | White is off white | White to off white | White to off white | White to off white |
| | viscous gel | viscous gel | viscous gel | viscous gel |
| pH (10% solution) | 6.08 | 6.09 | 6.07 | 6.02 |
| Viscosity (cps) | 1959 | 1913 | 1725 | 1875 |
| Time (Hrs) | Cumulative drug release (μg/20 ml) | | | |
| 0 | 0.0 | 0.0 | 0.0 | 0.0 |
| 2 | 0.0 | 1.6 | 1.8 | 3.3 |
| 4 | 10.1 | 13.9 | 13.6 | 16.2 |
| 6 | 24.6 | 28.7 | 25.1 | 29.0 |
| 8 | 39.3 | 45.8 | 42.7 | 46.1 |
| 12 | 79.7 | 88.6 | 80.3 | 80.9 |
| 24 | 148.7 | 190.0 | 171.3 | 162.0 |
| 30 | 191.7 | 236.0 | 208.4 | 189.0 |
| 48 | 247.7 | 296.4 | 277.5 | 237.4 |

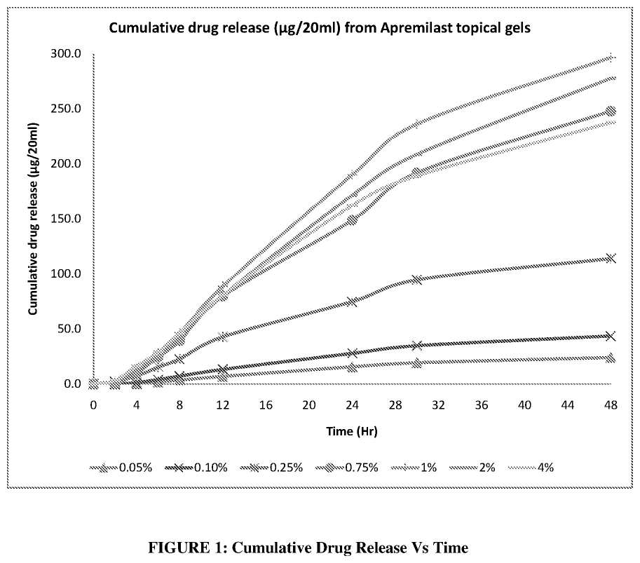

Based on the above Table 1 and Table 2 it was inferred that the drug release is linear with increased strength until the drug is in solution form as in Example 2 clear gels, whereas in case of Example 3 dispersive gels the drug release is more or less constant irrespective of strengths. Thus the present invention clear gels were found to be effective in providing a linear release of drug over a period of 48 hours even with a lesser amount of drug, however the higher amount of drug containing dispersive gels were found to be unclear or opaque due to the possible precipitation of the drug or presence of drug in dispersion forms in the formulation over time, thereby the drug release was flat or constant irrespective of a higher strength (as shown in FIG. 1).

The Drug samples were further evaluated in terms of other physical and biological parameters and where found to be satisfactory over conventional dosage forms and high strength compositions of Apremilast.

We claim:

1. A low dose clear topical composition of Apremilast comprising apremilast or its pharmaceutically acceptable salt in the range of 0.01-1.5% w/w with at least one solvent and one or more pharmaceutically acceptable excipient.

2. The topical composition as claimed in claim 1, wherein the said clear topical composition gives a linear and proportional drug release.

3. The topical composition as claimed in claim 1, wherein the said clear composition is a gel, paste, cream, ointment or lotion.

4. The topical composition as claimed in claim 1, wherein the said pharmaceutically acceptable excipient is selected from the group comprising of gelling agents, solvents, co-solvents, skin penetration enhancers, pharmaceutical surfactants, solubility enhancers or solubilizers, oil phase components, aqueous phase components, emulsifiers, moisturizers, antioxidants, vitamins, lubricants, preservatives, stabilizers, buffers, alkalizers or a mixture thereof.

5. The topical composition as claimed in claim 1, wherein the said pharmaceutical composition is suitable for topical application, comprising Apremilast or its pharmaceutically acceptable salt in lower amount capable of delivering therapeutically effective amounts in combination with pharmaceutically acceptable excipients for alleviating the symptoms of Psoriasis and associated inflammatory skin conditions.

6. The topical composition as claimed in claim 1, wherein the said composition characteristically provides enhanced drug delivery and skin permeation with minimal amount of Apremilast in the composition.

7. The topical composition as claimed in claim 1, wherein the said composition characteristically gives a proportionate increase in the amount of drug getting diffused across the diffusion layer, throughout the range of 0.01% to 1.5% apremilast w/w.

8. The topical composition as claimed in claim 1, wherein in the said composition the pharmaceutical excipient is a gelling agent and the ratio of Apremilast to gelling agent in the composition is 1:10 to 1:100.

9. The topical composition as claimed in claim 1, wherein the said pharmaceutically acceptable excipient is a penetration enhancer or solubilizer, wherein the solubilizer can be a single solubilizer or surfactant or a combination of solubilizer or surfactant.

10. A low dose topical composition of Apremilast with one or more pharmaceutically acceptable excipient with charactertic linear and dose proportionate enhanced diffusion, wherein the said composition is prepared by a process comprising of:
a. Dispensing Apremilast, carrier or solvents, gelling agent, humectant, methyl paraben and propyl paraben;
b. Weighing the required quantity of Apremilast and dissolving in required quantity of a suitable first solvent or carrier under stirring; wherein the stirring was continued until the complete solubilization of Apremilast;
c. Weighing required quantity of water and a suitable humectant and mixing together under continuous stirring;
d. Adding weighed quantity of a second solvent to the step c) mixture under stirring with continuous stirring for 10 min;
e. Adding suitable gelling agent to the step d) mixture under stirring and the stirring was continued for 2 hrs till complete solubilization of the gelling agent;
f. There after step b) drug solution was added to the step e) gel base under stirring;
g. Separately weighed quantity of methyl and propyl parabens were then dissolved in required quantity of a third solvent.
h. Then the step g) preservative solution was added to the step f) Apremilast gel under stirring, followed by continued stirring for 30 min; and
i. Finally the pH of the gel was adjusted in the range of pH 6.0-7.0 by adding 10% w/v NaOH solution;
wherein the solvent used in step a), b), d) and g) can be same or different solvents.
